(19) United States
(12) Patent Application Publication
(10) Pub. No.: US 2017/0071973 A1
(43) Pub. Date: Mar. 16, 2017

(54) EXTENDED SHELF LIFE WOUND HEALING FORMULATION

(71) Applicants: Mark DEACON-SHAW, Sydney (AU); Graham KELLY, Sydney (AU)

(72) Inventors: Mark DEACON-SHAW, Sydney (AU); Graham KELLY, Sydney (AU)

(21) Appl. No.: 15/309,356

(22) PCT Filed: May 7, 2015
(86) PCT No.: PCT/AU2015/050221

§ 371 Date: Nov. 7, 2016

Related U.S. Application Data
(60) Provisional application No. 61/989,980, filed on May 7, 2014.

(51) Int. Cl. A61K 31/716; A61K 9/06; A61K 47/02; A61K 47/32; A61K 47/10; A61K 47/18

(52) U.S. Cl. CPC A61K 31/716; A61K 47/10; A61K 47/18; A61K 47/02; A61K 47/32; A61K 9/06

(57) ABSTRACT

A composition for treatment of a dermal injury including a micro-particulate glucan (MPG); and a preservative in the form of an alcohol; wherein the MPG is provided in an amount effective for enabling the composition to induce wound healing or wound repair.

### FIELD OF THE INVENTION

The invention relates to wounds of dermal tissue and to extended shelf life gels and like formulations adapted for topical administration to dermal tissue wounds for treatment of same.

### BACKGROUND OF THE INVENTION

Reference to any prior art in the specification is not, and should not be taken as, an acknowledgment or any form of suggestion that this prior art forms part of the common general knowledge in Australia or any other jurisdiction or that this prior art could reasonably be expected to be ascertained, understood and regarded as relevant by a person skilled in the art.

The process of tissue repair within a dermal wound involves a complex set of biological responses that progressively result in the removal of damaged tissue, angiogenesis, fibrosis and epithelialisation. Under normal circumstances this complex interplay of many different biological responses involving a variety of tissue types occurs in a well-coordinated manner, ending finally in a remodelling process that should result in minimal scar formation and a return to a fully functioning dermis.

It is generally recognised that the key to this entire process is the wound macrophage. The entry of macrophages into the wound space is the critical first step in the initiation of the biological cascade that is the tissue repair process. These wound macrophages are activated by the presence of damaged tissue, resulting in the production of signalling cytokines such as TNF-α whose role it is to attract other pro-inflammatory cells such as neutrophils, NK cells and monocytes into the wound space, and then to initiate the activity of cells involved in angiogenesis, fibrosis and epithelialisation.

In fresh wounds in healthy individuals, the influx of wound macrophages into the wound space can normally be expected to occur in a timely manner and at levels that will ensure that the repair process proceeds without interruption to a normal completion.

There are however other forms of wound where the local environment is so adverse to macrophages as to discourage their presence and their activity. Those wounds typically have delayed wounding and are inclined to remain chronic and become indurated, sometimes never healing over the life of the individual. Examples of such wounds are decubitus ulcers, pressure sores, venous stasis ulcers, and diabetic ulcers.

In this knowledge, it can be seen therefore that any treatment modality for any sort of dermal wound must ensure that it does not impede the ability of macrophages to enter the wound space or to perform their normal function. Given the critical importance of these cells to the healing process, it readily can be seen that any treatment modality that did so impede macrophage activity would be highly detrimental to the outcome of the treatment.

In some circumstances micro-particulate glucan (MPG), a carbohydrate sourced from the yeast, Saccharomyces cerevisiae, is useful in some indications requiring wound repair. This has been demonstrated in incisional wounds in healthy animals and in animals where the healing process was deliberately impeded by the use of immunosuppressive drugs. In each of these cases, the glucan was instilled into the wound as a dry powder.

This high molecular weight sugar acts as an antigen to the innate immune system, stimulating a range of cells including macrophages, neutrophils and monocytes through binding to surface Dectin-1 and TLR2 receptors.

The glucan can be applied to the wound in a variety of forms, including a dry powder form as per the animal studies cited above. In the case of incisional wounds where closure of the wound is readily effected by way of, for example, suturing or bandaging, a dry powder form is adequate because closure of the wound ensures that the glucan is retained within the wound space.

However, in the case of open wounds such as, for example, trophic ulcers and skin following laser ablation therapy, a powder form would be inappropriate as it would both dry out the wound and be very difficult to achieve even distribution across the wound surface. In such cases, a liquid form is preferred because both of the ability to spread the active ingredient evenly over the wound space and the ability to be compatible with a moist environment. Examples of a liquid form are a gel and an ointment, but any other medium that provided a moist, flowable vehicle could be used.

Using the example of a gel preparation containing MPG, it is a requirement of health authorities that such preparations to be applied to open skin wounds would need either to be sterile or to have a very low microbial burden and be preserved to prevent growth of residual microbes in the preparation during storage.

Typically, preservatives used in topical preparations include chemicals such as parabens, phenoxyethanol and sodium hydroxymethylglycinate. However such chemicals are contra-indicated in the matter of preparations used in the treatment of dermal wounds because it is well known that they inhibit the activity of macrophages. It can be readily seen therefore that their use in the case of preparations where the active ingredient is MPG would be entirely contra-indicated because those chemical preservatives could be expected to offset the beneficial effect of the MPG in attracting macrophages into the wound space and in activating those cells that are critical to the healing process.

In certain cosmetic preparations where the use of chemical preservatives seeks to be avoided, an alternative preservative is ethanol (ethyl alcohol). Ethanol is also used in medical wipes and in many anti-bacterial hand sanitizer gels at a concentration of about 60% v/v as an antiseptic. Ethanol kills organisms by denaturing their proteins and dissolving their surface lipids and is effective against most bacteria and fungi and many viruses. In cosmetic preparations such as gels, the ethanol is used as a preservative rather than as an antiseptic and in that case has been found to be an adequate preservative at levels of between about 20-30% v/v.

Cosmetic preparations being preserved by ethanol are intended for use on intact skin. That is because ethanol is both an irritant and an inhibitor of immune function, effects that would be entirely contra-indicated in the case of open wounds or skin where the integrity of the epidermis had become damaged to the extent that the natural protective barrier of the skin is compromised.

As an irritant, ethanol reasonably could be expected to impede the effect of MPG. MPG itself is a pro-inflammatory, but the resulting inflammatory process is a carefully coordinated cellular process that progressively recruits the various layers of the tissue repair process. The sort of inflammation induced by ethanol is that associated with an acute tissue injury typified by the production of chemicals such as histamine that induce an acute inflammatory exudation (swelling) and which are designed to alert the organism to the presence of a damaging irritant. Such an inflammatory response is not associated with tissue repair, and the inflammatory exudate even impedes the innate immune capacity of skin. Ethanol has been shown to impede the recruitment of neutrophils to infected sites and to lead to reduced levels of the cytokine, TNF-alpha, a cytokine produced by macrophages and which is critical to the tissue repair process (Boe D M, Nelson S, Zhang P, Bagby G J. Acute ethanol intoxication suppresses lung chemokine production following infection with Streptococcus pneumoniae. J. Infect. Dis. 2001; 184(9):1134-1142; Boe D M, Nelson S, Zhang P, Quinton L, Bagby G J. Alcohol-induced suppression of lung chemokine production and the host defense response to Streptococcus pneumoniae. Alcohol. Clin. Exp. Res. 2003; 27(11):1838-1845; Quinton L J, Nelson S, Zhang P, Happel K I, Gamble L, Bagby G J. Effects of systemic and local CXC chemokine administration on the ethanol-induced suppression of pulmonary neutrophil recruitment. Alcohol. Clin. Exp. Res. 2005; 29(7):1198-1205).

Perhaps of greatest biological significance, however, exposure to ethanol has been shown to diminish the activity of macrophages, (Heinz R, Waltenbaugh C. Ethanol-consumption modifies dendritic cell antigen presentation in mice. Alcohol. Clin. Exp. Res. 2007; 31(10):1759-1771), including their ability to migrate (Lau A H, Thomson A W, Colvin B L. Chronic ethanol exposure affects in vivo migration of hepatic dendritic cells to secondary lymphoid tissue. Hum. Immunol. 2007; 68(7):577-585).

Taken together, these well-known characteristics of ethanol suggest that it would be contra-indicated to use ethanol as a preservative for a formulation where that formulation was to be used specifically on an open wound with the intention of accelerating the healing process in that wound.

As discussed, liquid forms of wound healing formulations are generally preferred for open wounds because these formulations enable the relevant wound healing active ingredient to be spread evenly over the wound space. Further they have compatibility with a moist environment such as an open wound.

One problem with gels and the like is that they are vulnerable to microbial contamination during formulation. Such contamination can limit the usage of the formulation in an open wound, especially as use of a contaminated formulation would ostensibly infect the open wound.

Further, it is not possible to sterilise a gel or the like, for example by autoclaving, without substantially destroying the desired properties of flow. Sterilisation prevents development of microbial contamination during storage. As preservatives such as those discussed above are generally considered to be contra indicated for open wounds, a problem remains as to how to extend the shelf life of a wound healing formulation such as a gel over a storage period i.e. how to ensure that the formulation remains free of microbial contamination over a storage period. The clear advantage of an extended shelf life product is that it avoids the need to formulate a wound healing formulation at the time of treatment of the injury.

There remains an urgent need for improvements in wound care, in particular in the area of liquid preparations used as a delivery vehicle for MPG intended to promote wound healing. In particular, a means needs to be found to preserve the preparation to prevent microbial growth during storage of the preparation, but such means must not either counteract the stimulatory effect of the MPG on wound macrophages, or conversely promote an inflammatory reaction by way of irritation so as to disturb the finely-balanced inflammatory cascade that is the tissue repair process.

### SUMMARY OF THE INVENTION

The invention seeks to address one or more of the above identified needs or limitations and in one embodiment the invention provides a composition for inducing wound healing including:

a micro-particulate glucan (MPG); and

a preservative in the form of an alcohol,

wherein the MPG is provided in an amount effective for enabling the composition to induce wound healing.

In another embodiment the invention provides a composition for treatment of a dermal injury including:

MPG in an amount of 0.05% to 1.0% (w/w) of the composition; and

ethanol in an amount of 5 to 15% (w/w) of the composition.

In a further embodiment there is provided a method of treating a skin wound including the step of administering a composition as described above to a skin wound.

In a further embodiment there is provided a use of a composition as described above for the treatment of a dermal injury.

In a further embodiment there is provided a kit including a plurality of unit doses of a composition as described above.

In a further embodiment there is provided a use of a kit as described above in a method as described above.

Further aspects of the present invention and further embodiments of the aspects described in the preceding paragraphs will become apparent from the following description, given by way of example and with reference to the accompanying drawings.

As used herein, except where the context requires otherwise, the term “comprise” and variations of the term, such as “comprising”, “comprises” and “comprised”, are not intended to exclude further additives, components, integers or steps.

### BRIEF DESCRIPTION OF THE DRAWINGS

FIG. 1: General structure of a microparticulate poly-(1,3)-beta-D-glucopyranosyl-(1,6)-beta-D-glucopyranose from Saccharomyces cerevisiae.

### DETAILED DESCRIPTION OF THE EMBODIMENTS

In our work described herein we have found that it is possible to provide a preservative effect to a gel formulation using an amount of ethanol that would not impede the wound healing effect of micro-particulate glucan. In more detail, as described below and exemplified herein, we have found that by use of ethanol and micro-particulate glucan, it is possible to extend the storage life of a gel for a period of up to about 30 months in the relevant storage conditions.

Further, we have found that the combination of micro-particulate glucan and ethanol in an amount that would not impede the wound healing effect of micro-particulate glucan has an anti-microbial effect. In more detail, as described below and exemplified herein, we have found that gel samples that were subjected to an antimicrobial effective test according to the USP<5122 methodology met USP<51> guidelines after a period of up to 28 days.

As described herein, the preservative effect at the relevant amount of ethanol arises from the presence of micro-particulate glucan and the ethanol. As exemplified herein, the preservative effect appears to arise from a synergistic interaction between the micro-particulate glucan and ethanol.

A number of important advantages arise from these findings. First as described herein, it becomes possible to provide for gel formulations including MPG having an extended storage life in the sense that across the relevant storage period the gels are effectively devoid of microbial contamination. This is achieved without substantially impacting on the viscosity of the gel. This avoids the need to prepare an MPG gel formulation at the time of wound therapy.

Second, as described herein, the combination of the ethanol and micro-particulate glucan effectively prevents the development of accidental or opportunistic infection in a gel formulation, whether that formulation is to be used immediately after formulation or stored. It is believed that this advantage is particularly relevant to treatment of open wounds with a gel where the very application of a gel to an open wound such as a burn or the like is associated with a risk of accidental infection.

These findings are surprising given that while the wound healing benefits of MPG were known, prior to the invention it had been widely understood that alcohol could not be used to preserve a composition for application to open wounds. Therefore, at the time of the invention, there was a complete paucity on the effect of MPG on alcohol—mediated tissue inflammation and immune cell inhibition.

Thus in one embodiment there is provided a composition for inducing wound healing including:

a micro-particulate glucan (MPG); and

a preservative in the form of an alcohol,

wherein the MPG is provided in an amount effective for enabling the composition to induce wound healing.

In another embodiment there is provided a composition including:

MPG in an amount of 0.05% to 1.0% (w/w), preferably 0.05% to 0.5% (w/w), preferably 0.05% to 0.1% (w/w) of the composition; and

ethanol in an amount of 5 to 15% (w/w) of the composition.

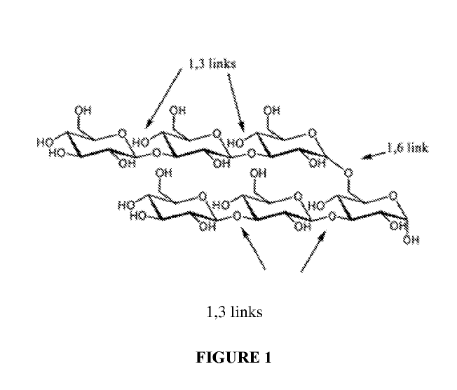

The composition of the invention includes MPG, also described as particulate β glucan. Glucans are oligosaccharides or polysaccharides composed predominantly or wholly of glucose. Glucans are widely distributed in nature, being found in the cell walls of a variety of plants, fungi and microorganisms. Typically the β glucan is a β (1,3)(1,6) glucan, more preferably a branched β (1,3)(1,6) glucan more preferably a poly-(1,3)-β-D-glucopyranosyl-(1,6)-β-D-glucopyranose. U.S. Pat. No. 6,242,594 describes the isolation of which glucan has the general structure as depicted in FIG. 1.

This substance typically has a molecular weight of between about 1 million and 2.2 million Daltons and is an amorphous powder slightly soluble in most aqueous and organic solvents but sparingly soluble in DMSO.

Whilst exemplified herein, those skilled in the art will appreciate that the scope of the present invention is not limited to the glucan described in U.S. Pat. No. 6,242,594 or a glucan produced in accordance with the methods described therein.

Generally the β glucan is provided in a composition in a therapeutically effective amount. This is generally an amount that enables the desired therapeutic outcome to be achieved when the composition is utilised for treatment of the dermal injury. The actual amount required in the composition of the invention depends on the therapeutic outcome to be obtained and the volume of the composition given in the administration. Generally the therapeutically effective amount is less than 10% (w/w) of the composition. In one embodiment, the amount is from about 0.01 to 1% (w/w) of the composition, preferably from about 0.01 to 0.15% (w/w) of the composition, more preferably about 0.05 to about 0.1% (w/w) of the composition, preferably about 0.1% (w/w) of the composition.

It will be understood that % (w/w) refers to mass of the relevant component as a proportion of the mass of the composition that contains it. For example, 0.1% (w/w) of the composition generally equates to 0.1 g of relevant component per 100 g of composition. Specifically, MPG in an amount of 0.1% w/w refers to 0.1 g MPG per 100 g of composition.

The particulate β glucan may be obtained from a range of biological sources including plants, fungi and micro-organisms. In one particularly preferred embodiment, the β glucan is derived from Saccharomyces cerevisiae.

The β glucan may be obtained by a range of processes known in the art. These include the isolation method described in U.S. Pat. No. 6,242,594.

Typically the composition does not include soluble β glucans. In these embodiments, and with respect to glucan content, the composition consists of, or consists essentially of particulate glucan, and especially particulate β glucan, such as micro particulate β glucan.

Importantly we have found that the shelf life of compositions containing particulate α glucan for use in treatment of dermal injury and/or wound healing can be maintained even at alcohol contents as low as 5 to 10% (w/w) of the composition. This is significant because it extends the scope of practical application of particulate β glucan compositions for treatment of wound healing insofar as enabling use of compositions that have been manufactured and stored for a significant time period before use. Thus it has been found that the preservative and/or sterilisation benefits of higher content alcohol compositions are also observed at lower alcohol contents.

A particular advantage of the compositions of the invention is that the lower alcohol content minimises the induction of stinging and/or burning sensations when the composition is applied to dermal injuries. This means that the compositions of the invention provide for a therapy that, upon administration to dermal injury is, in a relative sense, pain free.

The preservative in the form of the alcohol may generally have two functions, the first being to preserve the particulate β glucan enabling compositions containing the same to be stored for up to about 2 years in appropriate storage conditions, and the second being to provide some degree of sterilization to the composition thereby preventing growth or culture of micro-organisms in the composition. Any alcohol suitable for this purpose in the compositions of the invention may be used. Particularly useful examples of alcohols include ethyl alcohol otherwise known as ethanol.

In certain embodiments, the preservative is provided in an amount of generally less than 20% (w/w) of the composition, or from about 1 to about 20% (w/w) of the composition. Preferred ranges include from 5 to 15% (w/w) of the composition, preferably from about 7.5 to 12.5% (w/w) of the composition, preferably about 10% (w/w) of the composition. As described herein these amounts of alcohol may provide for a shelf life of the composition in storage conditions of up to about 2 years from manufacture.

It will be understood that the amounts of alcohol described herein are calculated on the basis of 100% alcohol. In more detail, ethanol at 10% (w/w) composition refers to an amount of 10 g of 100% ethanol per 100 g of composition.

The amount effective for enabling the composition to induce wound healing may be expressed in terms of the ratio of glucan/weight composition:alcohol/weight composition.

The preservative in the form of the alcohol may affect the pH of the compositions of the invention. Generally, as the alcohol content of the compositions decreases, the pH decreases. In turn, the pH impacts the viscosity of the composition. Generally, as the pH decreases, the viscosity of the composition reduces. In certain embodiments, the pH of the compositions of the invention is from about 5.0 to about 8.0, preferably from about 5.5 to about 7.5, more preferably from about 6.0 to about 7.5, more preferably from about 6.3 to about 7.3. In a particularly preferred embodiment of the invention, the pH of the composition is about 7.0.

The composition of the invention may be used for inducing healing of wound arising from dermal injury. A dermal injury may be any injury of the skin or underlying tissue including dermal layers, fat and subcutaneous tissue. Injuries of particular concern are those resulting in a breakage of the skin layer that exposes underlying tissue. The injury may arise from trauma, infection or from surgery. Where the injury arises from trauma, it may be a laceration, puncture abrasion, contusion or burn. The skin wound or lesion may be a surgical wound or result from physical damage, injury or trauma.

Where the injury arises from infection it may be a suppurative or non suppurative lesion. Where the injury arises from surgery it may be a surgical wound arising from surgical intervention such as incision, stapling or sewing, laser or chemical treatments, or insertion of a catheter or the like.

Generally the composition is applied to the dermal injury immediately after injury. Specifically, the inventor recognises that there is a direct relationship between induction of local immune inhibition at the wound site and time from injury whereby inhibition increases as time from injury increases. In one embodiment the composition is applied within 24 hours from injury, preferably within 12 hours from injury, preferably within 6 or 3 or 2 or 1 hour from injury.

In some embodiments it may be necessary to remove inflammatory agents from the site of injury before administration of the composition of the invention to the wound. This is particularly the case where the injury site is infected and/or contains an irritant, allergen or some other type of agent capable of providing for unwanted stimulation of innate immune cells. These agents may be removed by washing the wound site with a sterile, non inflammatory solution of a solution having a pH and salt content that is compatible with the relevant tissue, prior to application of the composition of the invention to the wound site. The wound site may be washed according to standard techniques thereby removing inappropriate stimuli for wound macrophages and other innate immune cells.

Where the dermal injury is in the form of a chronic wound, it may be necessary to surgically resect the chronically inflamed tissue to form a surgical wound and then to treat the surgical wound with the composition of the invention. In these embodiments, the objective is to remove tissue that has been subjected to local immune inhibition and to treat the adjacent tissue that has not been immune inhibited and that becomes accessible for contact with the composition of the invention when the chronically inflamed tissue has been removed.

Where the dermal injury is associated with infection, it may be necessary to remove microbial pathogens by antibiotic treatment or other drug therapy. As these chemotherapies may impact on immune inhibition of wound macrophages, it may be necessary to apply further treatment modalities. For example, it may be necessary for there to be a time delay between chemotherapy or antibiotic treatment and use of a composition of the invention to enable the local immune inhibition effects at the wound site arising either from infection or chemotherapy to be minimised before the composition of the invention is utilised. In another embodiment, particularly where the innate immune cells and/or wound macrophages are immune inhibited, it may be necessary to adjust the cytokine profile at the wound site to prime these cells, for example using GM CSF, IL-1 or other either before or at the time of administration of the composition of the invention.

The treatment of the dermal injury may involve remedying a condition or symptoms, preventing the establishment of a condition or disease, or otherwise preventing, hindering, retarding, or reversing the progression of a condition or disease or other undesirable symptoms in any way whatsoever. In one embodiment the treatment of dermal injury may result in any one or more of the following actions, processes or outcomes:

minimisation of inflammation

minimisation of fibrosis

deposition of collagen

differentiation of stem cells into functional dermal cells

reduction in scar formation

reversal of scarring or fibrotic lesions

angiogenesis

re-epithelialisation

granulation tissue formation.

The composition may be provided in the form of an intermediate composition which is then formulated to provide a therapeutic composition for administration to a dermal injury. An intermediate form of the composition may generally include the particulate β glucan; and the preservative in the form of an alcohol. Both of these components are as described above. The concentration of the glucan and alcohol in the intermediate may be greater than that found in the therapeutic composition to be applied to dermal injury in circumstances where the intermediate composition is to be diluted by addition of other components to it to form the therapeutic composition. Therefore, in certain embodiments the intermediate composition may include particulate β glucan in an amount of from 5 to 15% and a preservative in the form of an alcohol in an amount from 25 to 35% alcohol.

The process for forming the composition of the invention generally involves forming an aqueous suspension of micro-particulate β glucan and mixing it with an alcohol to form a preserved, sterilized therapeutic composition.

In the above described compositions, the agent may be administered in the form of pharmaceutically acceptable nontoxic salts, such as acid addition salts or metal complexes, e.g. with zinc, iron or the like (which are considered as salts for purposes of this application).

Illustrative of such acid addition salts are hydrochloride, hydrobromide, sulphate, phosphate, maleate, acetate, citrate, benzoate, succinate, malate, ascorbate, tartrate and the like.

Examples of pharmaceutically acceptable carriers or diluents are demineralised or distilled water; saline solution; vegetable based oils such as peanut oil, safflower oil, olive oil, cottonseed oil, maize oil, sesame oil, arachis oil or coconut oil; silicone oils, including poly siloxanes, such as methyl polysiloxane, phenyl polysiloxane and methylphenyl polysolpoxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethylcellulose; lower alkanols, for example ethanol or iso-propanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrridone; agar; carrageenan; gum tragacanth or gum acacia, and petroleum jelly. Typically, the carrier or carriers will form from 10% to up to about 90% (w/w) of the composition.

Topical formulations typically comprise an active ingredient together with one or more acceptable carriers, and optionally any other therapeutic ingredients. Formulations suitable for topical administration include liquid or semi-liquid preparations suitable for penetration through the skin to the site of where treatment is required, such as liniments, lotions, creams, ointments or pastes, and drops.

Lotions according to the present invention include those suitable for application to the skin or eye. An eye lotion may comprise a sterile aqueous solution optionally containing a bactericide and may be prepared by methods similar to those described above in relation to the preparation of drops. Lotions or liniments for application to the skin may also include an agent to hasten drying and to cool the skin, such as an acetone, and/or a moisturiser such as glycerol, or oil such as castor oil or arachis oil.

Creams, ointments or pastes according to the present invention are semi-solid formulations of the active ingredient for external application. They may be made by mixing the active ingredient in finely-divided or powdered form, alone or in solution or suspension in an aqueous or non-aqueous fluid, with a greasy or non-greasy basis. The basis may comprise hydrocarbons such as hard, soft or liquid paraffin, glycerol, beeswax, a metallic soap; a mucilage; an oil of natural origin such as almond, corn, arachis, castor or olive oil; wool fat or its derivatives, or a fatty acid such as stearic or oleic acid together with an alcohol such as propylene glycol or macrogols.

Pharmaceutical forms that are suitable for injection include sterile aqueous solutions (where water soluble) or dispersions and sterile powders for the extemporaneous preparation of sterile injectable solutions. It must be stable under the conditions of manufacture and storage and must be preserved against the contaminating action of microorganisms such as bacteria and fungi. The carrier can be a solvent or dispersion medium containing, for example, water, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of superfactants. The prevention of the action of microorganisms can also be brought about by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

By way of example only, the composition for topical administration may comprise, as exemplified herein, glucan in microparticulate form, in a composition comprising 10% (w/w) ethanol, triethanolamine, Carbopol® 980 NF, titanium dioxide and purified water. The resulting composition may be a highly viscous, aqueous gel suitable for topical administration.

Sterile injectable solutions are prepared by incorporating the active compounds in the required amount in the appropriate solvent with various of the other ingredients enumerated above, as required, followed by filtered sterilisation. Generally, dispersions are prepared by incorporating the various sterilised active ingredient into a sterile vehicle which contains the basic dispersion medium and the required other ingredients from those enumerated above, hi the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

In one embodiment, the composition may be provided in the form enabling spraying of the composition onto a dermal injury. The composition may be formulated so as to have properties of flow enabling spraying. When sprayed onto skin, the composition may then take on the form of a gel.

The present invention contemplates combination therapies, wherein agents as described herein are coadministered with other suitable agents which may facilitate the desired therapeutic or prophylactic outcome. For example, one may seek to maintain ongoing anti-inflammatory therapies in order to control the incidence of inflammation whilst employing agents in accordance with embodiments of the present invention. By “coadministered” is meant simultaneous administration in the same formulation or in two different formulations via the same or different routes or sequential administration by the same or different routes. By “sequential” administration is meant a time difference of from seconds, minutes, hours or days between the administration of the two types of molecules. These molecules may be administered in any order.

In another embodiment the glucan is administered in the form of a dressing or bandage into which a composition according to the invention has been incorporated. The compositions may be impregnated into transdermal patches, plasters, and wound dressings such as bandages or hydrocolloid dressings, preferably in liquid or semi-liquid form. By way of example only, topically applied compositions in accordance with the present invention may be formulated into, or with, face masks and scrubs, conditioning products such as lotions and creams, oils, shaving products such as creams and gels, skin washes, foams, bath and shower preparations such as oils and gels, moisturising products such as lotions, creams, gels and foams, anti-wrinkle products and anti-ageing products.

In a further embodiment there is provided a method of treating a dermal injury including the step of administering a composition as described above to a dermal injury.

In a further embodiment there is provided a use of a composition as described above for the treatment of a dermal injury.

In a further embodiment there is provided a kit including a plurality of unit doses of a composition as described above.

In a further embodiment there is provided a use of a kit as described above in a method as described above.

It will be understood that the specific dose level of a composition of the invention for any particular individual will depend upon a variety of factors including, for example, the activity of the specific agents employed, the age, body weight, general health and diet of the individual to be treated, the time of administration, rate of excretion, and combination with any other treatment or therapy. Single or multiple administrations can be carried out with dose levels and pattern being selected by the treating physician. A broad range of doses may be applicable. Considering a patient, for example, from about 0.1 mg to about 1 mg of agent may be administered per wound surface area. Dosage regimens may be adjusted to provide the optimum therapeutic response. For example, several divided doses may be administered daily, weekly, monthly or other suitable time intervals or the dose may be proportionally reduced as indicated by the exigencies of the situation.

More typically, an effective dose range is expected to be in the range about 1.0 mg to about 200 mg per kg body weight per 24 hours; about 1.0 mg to about 100 mg per kg body weight per 24 hours; about 1.0 mg to about 50 mg per kg body weight per 24 hours; about 1.0 mg to about 25 mg per kg body weight per 24 hours; about 5.0 mg to about 50 mg per kg body weight per 24 hours; about 5.0 mg to about 20 mg per kg body weight per 24 hours; about 5.0 mg to about 15 mg per kg body weight per 24 hours.

It will be understood that the invention disclosed and defined in this specification extends to all alternative combinations of two or more of the individual features mentioned or evident from the text or drawings. All of these different combinations constitute various alternative aspects of the invention.

### EXAMPLES

### Example 1

1. Aim/Method

The aim of this study is to establish the stability of 1.0% micro-particulate glucan and 0.1% micro-particulate glucan, with respect to Appearance, Viscosity, pH, Microbiological testing and preservative Efficacy testing.

2. Study Details

The Study details involves the description of the raw material used, formulation, packaging and storage of the material

2.1 Raw Materials Used

| Raw Material Name | Grade |
|---|---|
| Micro-particulate glucan | Made I house (Low Sugar Instant |
| | Dry Yeast) |
| Titanium Dioxide | Ph. Eur |
| Water for Injections | BP purified |
| Carbopol 981 | Not specified |
| Ethanol 1005 | 100% AR |
| AR | 100% HG(BP) |
| Triethanolamine | Not specified |

2.2 Formulation

2.2.1 The Formulations for 1.0% Micro-Particulate Glucan at 4° C.

| Description | QTY required |
|---|---|
| Micro-particulate glucan | 150.0 g |
| Titanium Dioxide | 150.0 g |
| Water for Injection | 11925.0 g |
| Carbopol 981 | 300.0 g |
| Ethanol 100% AR | 2250.0 g |
| Triethanolamine | 225.0 g |

2.2.2 The Formulations for 0.1% Micro-Particulate Glucan at 4° C.:

| Description | QTY required |
|---|---|
| Micro-particulate glucan | 15.0 g |
| Titanium Dioxide | 15.0 g |
| Water for Injections | 12060.0 g |
| Carbopol 981 | 300.0 g |
| Ethanol 100% AR | 2250.0 g |
| Triethanolamine | 225.0 g |

2.2.3 The Formulations for 1.0% Micro-Particulate Glucan at 25° C.

| Description | QTY required |
|---|---|
| Micro-particulate glucan | 150.0 g |
| Water for Injections | 12070.0 g |
| Carbopol 981 | 300.0 g |
| Ethanol 100% AR | 2250.0 g |
| Triethanolamine | 225.0 g |

(i) Packaging

SPPM-1063 White 30 ml polypropylene cosmetic jar for Micro-particulate glucan

SPPM-1064 Casca seal to fit 30 ml Micro-particulate glucan jar

SPPM-1065 Dome cap to fit 30 ml Micro-particulate glucan jar

(ii) Storage Conditions

All jars are refrigerated at 4° C.±3° C., the fridge conditions are monitored by means of a data logger which measure temperature. Humidity is also recorded.

Batch 1GG03-008 stored at 25±3° C. incubator where the temperature is monitored by means of a data logger which measures both temperature and humidity. The humidity for this incubator is below the specified ICH guidelines of 60%±5% RH. The temperature is within range of ±3° C.

2.3 Study Criteria

The study is to provide sufficient evidence that 1.0% and 0.1% Micro-particulate glucan is stability indicating for the 6 month duration, showing:
little to no deterioration in the ethanol content
the chemical testing complies to the report specifications
the micro testing complies to the report specifications

2.4 Testing Regime

This study does not follow the full ICH stability guidelines in that intermediate and accelerated time points were not commenced for majority of the batches due to this facility being a Pilot facility.

3. Summary and Interpretation of Data

TABLE 3.1.1
Stability Data captured for all 1.0% Micro-particulate glucan batches using Titanium Dioxide at 4° C.

| | | | | Appearance: | | | | | |
|---|---|---|---|---|---|---|---|---|---|
| | | | Date | Viscous | Ethanol | | Viscosity^{~}100000 | Preservative | |
| Batch No | Time Point | Temp | Completed | opague gel | content/% | pH: 5.5-6.5 | cps | Efficacy test | Micro tests |
| 1GG04-010 | 0 | 4 | 17 May 2004 | complies | 11.8-13.5% | 5.57-5.72 | >100000 cps | complies | complies |
| 1GG04-010 | 3 | 4 | 14 Jul. 2004 | complies | 14.40% | 5.55 | >90300 cps | complies | complies |
| 1GG04-010 | 6 | 4 | 15 Oct. 2004 | complies | 12.30% | 5.72 | >90300 cps | complies | complies |
| 1GG04-010 | 9 | 4 | 18 Mar. 2005 | complies | 12.00% | 5.47 | >90300 cps | complies | complies |
| 1GG04-010 | 12 | 4 | 10 May 2005 | complies | 11.80% | 5.45 | >90300 cps | complies | complies |
| 1GG04-012 | 0 | 4 | 15 Dec. 2005 | complies | 13.30% | 5.89 | >90300 cps | complies | complies |
| 1GG04-012 | 3 | 4 | 22 Feb. 2005 | complies | 12.40% | 5.6 | >90300 cps | complies | complies |
| 1GG04-012 | 6 | 4 | 1 Jun. 2005 | complies | 12.50% | 5.59 | >90300 cps | complies | complies |
| 1GG04-012 | 9 | 4 | 6 Sep. 2005 | complies | 11.60% | 5.52 | 114200 | complies | complies |
| 1GG04-013 | 0 | 4 | 15 Dec. 2005 | complies | 13.90% | 5.94 | >90300 cps | complies | complies |
| 1GG04-013 | 3 | 4 | 14 Apr. 2005 | complies | 13.70% | 5.66 | >90300 cps | complies | complies |
| 1GG04-013 | 6 | 4 | 1 Jun. 2005 | complies | 12.90% | 5.54 | >90300 cps | complies | complies |
| 1GG05-015 | 0 | 4 | 23 Mar. 2005 | complies | 13.70% | 5.57 | >90300 cps | complies | complies |
| 1GG05-015 | 3 | 4 | 1 Jun. 2005 | complies | 13.70% | 5.66 | >90300 cps | complies | complies |
| 1GG05-015 | 6 | 4 | 5 Sep. 2005 | complies | 13.20% | 5.53 | 114600 cps | complies | complies |

TABLE 3.1.2
Stability Data captured for all 0.1% Micro-particulate glucan batches using Titanium Dioxide at 4° C.

| | | | Data | Appearance: | Ethanol | | Viscosity^{~}100000 | Preservative | |
|---|---|---|---|---|---|---|---|---|---|
| Batch No | Time Point | Temp | Completed | opague gel | content/% | pH: 5.5-6.5 | cpa | Efficacy | Micro tests |
| 1GA04-009 | 0 | 4 | 17 May 2005 | complies | 12.8-13.4% | 5.62-5.77 | >100000 cps | complies | complies |
| 1GA04-009 | 3 | 4 | 14 Jul. 2005 | complies | 13.90% | 5.51 | >903000 cps | complies | complies |
| 1GA04-009 | 6 | 4 | 15 Oct. 2004 | complies | 12.30% | 5.61 | >903000 cps | complies | complies |
| 1GA04-010 | 0 | 4 | 22 Oct. 2004 | complies | 12.65-13.9% | 5.6-5.7 | >903000 cps | complies | complies |
| 1GA04-010 | 3 | 4 | 5 Jan. 2005 | complies | 13.00% | 5.57 | >903000 cps | complies | complies |
| 1GA04-010 | 6 | 4 | 5 Apr. 2005 | complies | 12.10% | 5.53 | >903000 cps | complies | complies |
| 1GA04-011 | 0 | 4 | 15 Dec. 2004 | complies | 14.20% | 5.94 | >903000 cps | complies | complies |
| 1GA04-011 | 3 | 4 | 22 Feb. 2005 | complies | 13.3% | 5.59 | >903000 cps | complies | complies |
| 1GA04-011 | 6 | 4 | 1 Jun. 2005 | complies | 13.1% | 5.54 | >903000 cps | complies | complies |
| 1GA04-011 | 9 | 4 | 5 Sep. 2005 | complies | 12.60% | 5.45 | >903000 cps | complies | complies |

TABLE 3.1.3
Stability Data captured for all 1.0% Micro-particulate glucan no Titanium Dioxide at 25° C.

| | | | | Appearance: | Ethanol | | Viscosity^{~}100000 | Preservative | |
|---|---|---|---|---|---|---|---|---|---|
| Batch No | Time Point | Temp | DateCompleted | opague gel | content/% | pH: 5.5-6.5 | cpa | Efficacy | Micro tests |
| 1GG03-008 | 0 | 4 | 14 Nov. 2003 | complies | 13.3-14.0% | 5.57 | >1000000 cps | complies | complies |
| 1GG03-008 | 3 | 25 | 11 Feb. 2004 | complies | nr | 0 | nr | complies | complies |
| 1GG03-008 | 6 | 25 | 30 May 2004 | complies | 13.7% | 5.87 | >90300 cps | complies | complies |
| 1GG03-008 | 18 | 25 | 30 May 2005 | complies | 14.3% | 5.54 | >90300 cps | complies | complies |

Micro Testing involves: TAPC <10 cfu/g; Yeasts and Moulds <10 cfu/g; S. Aureus ND/g; E. coli ND/g; Ps. aeruginosa ND/g; Enterobacteriaceae <10 cfu/g.

Discussion

1.0% Micro-Particulate Glucan

Extension of expiry requires a minimum of three batches. Batches: 1GG04-010; 1GG04-012, 1GG04-013 and 1GG05-15 supports extension of expiry for 6 months as all results captured for stability are compliant with the test report specifications. All other batches support the three month stability of 1.0% Micro-particulate glucan.

Only one batch was put on 25° C. stability for an 18 month period, of which only 3, 6 and 18 months were tested. From the data in Table 3.1.3 the data obtained is stability indicating for 18 months with a better ethanol content and pH reading than the 1.0% Micro-particulate glucan stored at 4° C.

0.1% Micro-Particulate Glucan

The batches above in Table 3.1.2 support extension of expiry of 6 months as all results captured over the period are compliant with the test report specifications.

4. Conclusions

The study provided sufficient evidence that 1.0% and 0.1% Micro-particulate glucan is stability indicating for the 6 month duration, showing the microbiological testing compliant with the specifications set.

It should be noted that for the 1.0% and 0.1% Micro-particulate glucan both show deterioration of the ethanol content over the stability period which is proportional to the drop in pH results obtained.

To conclude 1.0% and 0.1% both can be given an extension of expiry of 6 months.

### Example 2

1. Aim

This study is to determine the minimum level of Ethanol required for Antimicrobial Effectiveness for the 1% Micro-particulate glucan gel and the Micro-particulate glucan Placebo.

2. Scope

This experiment covers five microorganisms over a 28 day period of inoculation for the 1% Micro-particulate glucan gel and the Micro-particulate glucan Placebo.

3. Packaging:

QC

White 30 ml polypropylene cosmetic jar for Glucan

4. Safety Precautions

Samples to be aseptically sampled (as per sample requirement table above) by trained production staff. Staff are to wear Tyveks, Racal respirators, shoe covers and a hair net for this procedure to protect both themselves and the product.

5. Procedure

The concentrations to be manufactured are 0%, 5%, 10% and 15% ethanol content in both the 1% Micro-particulate glucan gel and the Placebo.

The samples for Placebo and 1% Micro-particulate glucan gel with varying concentrations are to be prepared by production and a report is to be attached to this protocol.

These samples are to be sent to AMS using the USP<51> method for Antimicrobial Effectiveness Test.

The five microorganisms to be used are: S. aureus (ATCC No 6538), P. aueruginosa (ATCC No 9027); C. albicans (ATCC No 10231), A. niger (ATCC No 16404) and E. coli (ATCC No 8739)

The Duration of this study is 28 days.

6. Acceptance Criteria

USP<51>

Bacteria: NLT 2 log reduction from the initial count at 14 days, and no increase from the 14 days counts at 28 days

Yeast & Moulds: No increase from the initial calculated count at 14 and 28 days.

EU Pharm.

Bacteria: NLT 2 log reduction at 2 days and NLT 3 log reduction a 7 days and no increase at 28 days.

Yeast & Moulds: NLT 2 log reduction from the initial count at 14 days, and no increase from the 14 days counts at 28 days

7. Results

TABLE 7.1
pH Reading
pH Reading (5.5-6.5)

| | | 1% Micro-particulate | |
|---|---|---|---|
| Timepoint | Concentrations | glucan gel | Placebo |
| T = 0 | 0% | 5.3 | 5.1 |
| | 5% | 5.3 | 5.2 |
| | 10% | 5.4 | 5.3 |
| | 15% | 5.6 | 5.5 |

From the data tabulated above the pH reading is within the specified limits for the 15% ethanol content present in both the 1% glucoprime gel and the placebo, which suggests that the quantity of ethanol present is proportional to the pH reading. A lower content of ethanol would yield a lower pH.

TABLE 7.2
Viscosity Readings
Viscosity Reading aprox 100000 cps

| | | 1% microparticulate | |
|---|---|---|---|
| Timepoint | Concentrations | glucan gel | Placebo |
| T = 0 | 0% | 87,400 cps | 67,400 cps |
| | 5% | 87,200 cps | 71,400 cps |
| | 10% | 81,000 cps | 71,400 cps |
| | 15% | 82,000 cpa | 72,400 cpa |

From the data seen above the viscosity readings obtained from both the 1% glucoprime gel and the placebo both show low readings compared to what is normally expected (>90000 cps). On visual inspection there is no distinct difference from the formulation gels and placebo when compared to a production sample batch 1GG06-01.9.

Antimicrobial Effectiveness for 1% Micro-particulate
glucan Gel with various concentrations of Ethanol

| Ethanol | Time | | | | | |
|---|---|---|---|---|---|---|
| Content | Point | S. aureus | P. aeruginosa | E. coli | C. albicans | A. niger |
| 0% | Inoculum | 8.6 × 10⁵ | 1.0 × 10⁶ | 5.9 × 10⁵ | 9.2 × 10⁵ | 3.8 × 10⁵ |
| | cfu/ml | | | | | |
| | 0 hour | * | * | * | * | * |
| | 14 days | * | * | * | * | * |
| | 28 days | * | * | * | * | * |
| 5% | Inoculum | 8.6 × 10⁵ | 1.0 × 10⁶ | 5.9 × 10⁵ | 9.2 × 10⁵ | 3.8 × 10⁵ |
| | cfu/ml | | | | | |
| | 0 hour | 3.4 × 10⁵ | 2.8 × 10⁵ | 4.2 × 10⁵ | 4.4 × 10⁵ | 3.6 × 10⁵ |
| | 14 days | <10 | <10 | <10 | 2.5 × 10¹ | 2.7 × 10⁵ |
| | 28 days | <10 | <10 | <10 | <10 | 1.6 × 10⁵ |
| 10% | Inoculum | 8.6 × 10⁵ | 1.0 × 10⁶ | 5.9 × 10⁵ | 9.2 × 10⁵ | 3.8 × 10⁵ |
| | cfu/ml | | | | | |
| | 0 hour | 6.8 × 10⁵ | 5.2 × 10⁵ | 5.8 × 10⁵ | 4.1 × 10⁵ | 4.4 × 10⁵ |
| | 14 days | <10 | <10 | <10 | <10 | 1.4 × 10⁴ |
| | 28 days | <10 | <10 | <10 | <10 | 1.8 × 10² |
| 15% | Inoculum | 8.6 × 10⁵ | 1.0 × 10⁶ | 5.9 × 10⁵ | 9.2 × 10⁵ | 3.8 × 10⁵ |
| | cfu/ml | | | | | |
| | 0 hour | 7.6 × 10⁵ | 6.1 × 10⁵ | 5.0 × 10⁵ | 4.3 × 10⁵ | 3.4 × 10⁵ |
| | 14 days | <10 | <10 | <10 | <10 | <10 |
| | 28 days | <10 | <10 | <10 | <10 | <10 |

Note:
* indicates contamination of the sample with Chryseomonas Luteola, Micrococcus spp; and bacillus spp.

Antimicrobial Effectiveness for 1% Micro-particulate
glucan Placebo with various concentrations of Ethanol

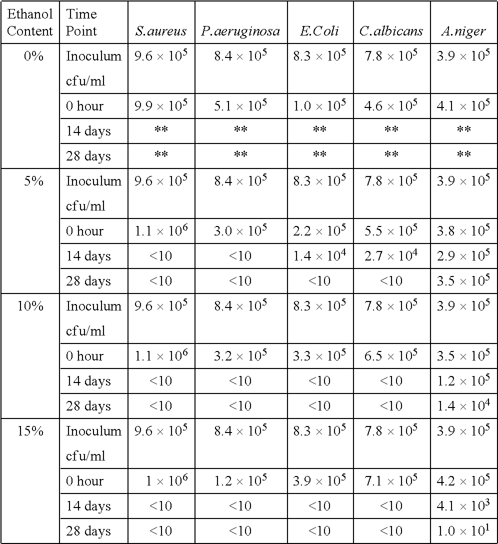

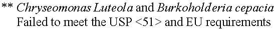

Conclusion

The 1% Micro-particulate glucan gel containing 5%, 10% and 15% of ethanol complies to the USP<51> Antimicrobial Effectiveness Test. Only the 10% and 15% placebo complies to the USP<51> Antimicrobial effectiveness Test requirements, the 5% placebo failed at the 14 day count. This suggests a synergistic relationship between the ethanol and micro-particulate glucan. Both the placebo and 1% Micro-particulate glucan gel failed in the absence of ethanol, which suggest that this formulation performs better in the presence of ethanol with a minimum requirement of 10% ethanol content for the formulation to meet with the USP<51> requirements. However, the pH limit will need to be revised as the pH range will be lower than the current specifications if 10% ethanol is added to the formulation.

Only Micro-particulate glucan and placebo gels containing 15% ethanol comply to both the USP<51> and the EU requirements of greater than 2 log reduction at 14 days and no increase at 28 days for fungi and no increase for bacteria at 28 days.

### Example 3

This example provides stability data at 29 months after formulation in Table 8.

TABLE 8.1
Placebo gel (no Micro-particulate glucan)

| | | | | Time(months) product stored at 25° C./60% RH | | | | | | | |
|---|---|---|---|---|---|---|---|---|---|---|---|
| | Test method | Specifications | | 0 | | 12 | | 18 | | 29 | |
| Test description | | | | | | | | | | | |
| Appearance | Visual | Viscous off | | Viscous off | | Viscous off | | Not tested | | Viscous off | |
| | examination | white gel | | white gel | | white gel | | | | white gel | |
| Identification & assay | Total hexose (%) | Confirms absence | | Confirms absence | | Confirms absence | | Confirms absence | | Confirms absence | |
| | | of MPG | | of MPG | | of MPG | | of MPG | | of MPG | |
| Viscosity | Chemika (pop) 43 | >50000 | cps | 99800 | | 89400 | | 82000 | | 88000 | |
| | (cps) | | | | | | | | | | |
| pH | USP<791> | 6.8 ± 0.5 | | 7.0 | | 6.9 | | 6.9 | | 6.8 | |
| Ethanol | USP<467> (%) | 10 ± 0.3% | | 9.5 | | 9.5 | | 9.6 | | 9.7 | |
| Bacterial endotoxins | Pyrogene | <15 | EU/g | N/A | | <5.0 | EU/g | <5.0 | EU/g | <5.0 | EU/g |
| | endotoxin test | | | | | | | | | | |
| Deliverable tube | BP Appendix XII | >10 | g | N/A | | 12 | | 12 | | N/A | |
| content | M (g) | | | | | | | | | | |
| Bioassay | MPG potency | report result | | N/A | | <15.6 | pg/ml | <15.6 | pg/ml | <15.6 | pg/ml |
| | assay | | | | | | | | | | |
| Microbiological test | | | | | | | | | | | |
| TVAC | USP <61&62> | <10 | cfu/g | <10 | cfu/g | <10 | cfu/g | <10 | cfu/g | <10 | cfu/g |
| Yeast | | <10 | cfu/g | <10 | cfu/g | <10 | cfu/g | <10 | cfu/g | <10 | cfu/g |
| Moulds | | <10 | cfu/g | <10 | cfu/g | <10 | cfu/g | <10 | cfu/g | <10 | cfu/g |
| Psuedomonas | | ND/10 | g | ND/10 | g | ND/10 | g | ND/5 | g | ND/10 | g |
| S. Aureus | | ND/10 | g | ND/10 | g | ND/10 | g | ND/5 | g | ND/10 | g |
| E. coli | | ND/10 | g | ND/10 | g | ND/10 | g | ND/5 | g | ND/10 | g |
| Salmonella spp | | ND/10 | g | ND/10 | g | ND/10 | g | ND/5 | g | ND/10 | g |

| | | | | | | | | |
|---|---|---|---|---|---|---|---|---|
| Enterobacteriaceae | <10² | cfu/g | <10 | cfu/g | <10 | cfu/g | N/A | N/A |

TABLE 8.2
1.0% Micro-particulate glucan

| | | | | Time(months) product stored at 25° C./60% RH | | | | | | | |
|---|---|---|---|---|---|---|---|---|---|---|---|
| | Test method | Specifications | | 0 | | 12 | | 18 | | 29 | |
| Test description | | | | | | | | | | | |
| Appearance | Visual | Viscous off | | Viscous off | | Viscous off | | Not tested | | Viscous off | |
| | examination | white gel | | white gel | | white gel | | | | white gel | |
| Identification & assay | Total hexose | 80-120% of | | 111 | | 106 | | 102 | | 112 | |
| | | label claim | | | | | | | | | |
| Viscosity | Chemika (pop) 43 | >50000 | cps | 142400 | | 127000 | | 123000 | | 115000 | |
| | (cps) | | | | | | | | | | |
| pH | USP<791> | 6.8 ± 0.5 | | 6.9 | | 6.8 | | 6.7 | | 6.7 | |
| Ethanol | USP<467> (%) | 10 ± 0.3 | | 9.7 | | 9 | | 9.4 | | 9.5 | |
| Bacterial endotoxins | Pyrogene | <15 | EU/g | N/A | | N/A | | N/A | | No Valid | |
| | endotoxin test | | | | | | | | | Results | |
| Content uniformity | USP <905> | complies with | | N/A | | N/A | | N/A | | N/A | |
| | | monograph | | | | | | | | | |
| Deliverable tube | BP Appendix XII | >10 | g | N/A | | 11 | g | 12 | g | N/A | |
| content | M (g) | | | | | | | | | | |
| Bioassay | MPG potency | report result | | N/A | | >1000.00 | pg/ml | >1000.00 | pg/ml | >1000.00 | pg/ml |
| | assay | | | | | | | | | | |
| Microbiological test | | | | | | | | | | | |
| TVAC | USP <61&62> | <10 | cfu/g | <10 | cfu/g | <10 | cfu/g | <10 | cfu/g | <10 | cfu/g |
| Yeast | | <10 | cfu/g | <10 | cfu/g | <10 | cfu/g | <10 | cfu/g | <10 | cfu/g |
| Moulds | | <10 | cfu/g | <10 | cfu/g | <10 | cfu/g | <10 | cfu/g | <10 | cfu/g |
| Psuedomonas | | ND/10 | g | ND/10 | g | ND/10 | g | ND/5 | g | ND/10 | g |
| S. Aureus | | ND/10 | g | ND/10 | g | ND/10 | g | ND/5 | g | ND/10 | g |
| E. coli | | ND/10 | g | ND/10 | g | ND/10 | g | | | ND/10 | g |
| Salmonella spp | | ND/10 | g | ND/10 | g | ND/10 | g | ND/5 | g | ND/10 | g |

| | | | | | | | | |
|---|---|---|---|---|---|---|---|---|
| Enterobacteriaceae | <10² | cfu/g | <10 | cfu/g | <10² | cfu/g | N/A | N/A |

TABLE 8.3
Micro-particulate glucan 0.1%

| | | | | Time(months) product stored at 25° C./60% RH | | | | | | | |
|---|---|---|---|---|---|---|---|---|---|---|---|
| | Test method | Specifications | | 0 | | 12 | | 18 | | 29 | |
| Test description | | | | | | | | | | | |
| Appearance | Visual | Viscous off | | Viscous off | | Viscous off | | Not Tested | | Viscous | |
| | examination | white gel | | white gel | | white gel | | | | white gel | |
| Identification & assay | Total hexose | 80-120% of | | Invalid Result | | 91 | | 117 | | 106 | |
| | | label claim | | | | | | | | | |
| Viscosity | Chemika (pop) 43 | >50000 | cps | 95800 | | 84400 | | 81200 | | 72000 | |
| | (cps) | | | | | | | | | | |
| pH | USP<791> | 6.8 ± 0.5 | | 7 | | 6.83 | | 6.9 | | 6.7 | |
| Ethanol | USP<467> (%) | 10 ± 3% | | 9.6 | | 9.4 | | 9.6 | | 9.8 | |
| Bacterial endotoxins | Pyrogene | <15 | eu/G | N/A | | N/A | | N/A | | No Valid | |
| | endotoxin test | | | | | | | | | Results | |
| Content uniformity | USP<905> | complies with | | N/A | | N/A | | N/A | | N/A | |
| | | monograph | | | | | | | | | |
| Deliverable tube | BP Appendix XII | >10 | g | N/A | | 13 | | 13 | | N/A | |
| content | M (g) | | | | | | | | | | |
| Bioassay | MPG potency | report result | | N/A | | 91.8 | pg/ml | 117.98 | pg/ml | 130.71 | pg/ml |
| | assay | | | | | | | | | | |
| Microbiological test | | | | | | | | | | | |
| TVAC | USP <61&62> | <10 | cfu/g | <10 | cfu/g | <10 | cfu/g | <10 | cfu/g | <10 | cfu/g |
| Yeast | | <10 | cfu/g | <10 | cfu/g | <10 | cfu/g | <10 | cfu/g | <10 | cfu/g |
| Moulds | | <10 | cfu/g | <10 | cfu/g | <10 | cfu/g | <10 | cfu/g | <10 | cfu/g |
| Psuedomonas | | ND/10 | g | ND/10 | g | ND/10 | g | ND/5 | g | ND/10 | g |
| S. Aureus | | ND/10 | g | ND/10 | g | ND/10 | g | ND/5 | g | ND/10 | g |
| E. coli | | ND/10 | g | ND/10 | g | ND/10 | g | ND/5 | g | ND/10 | g |
| Salmonella spp | | ND/10 | g | ND/10 | g | ND/10 | g | ND/5 | g | ND/10 | g |

| | | | | | | | | |
|---|---|---|---|---|---|---|---|---|
| Enterobacteriaceae | <10 ² | cfu/g | <10 | cfu/g | <10 | cfu/g | N/A | N/A |

1. A composition for treatment of a dermal injury including:
a micro-particulate glucan (MPG); and
a preservative in the form of an alcohol;
wherein the MPG is provided in an amount effective for enabling the composition to induce wound healing or wound repair.

2. The composition of claim 1 wherein the MPG is provided in an amount of from about 0.01 to 1% (w/w) of the composition.

3. The composition of claim 1 wherein the preservative is provided in an amount of from about 1 to about 30% (w/w) of the composition.

4. The composition of claim 1 wherein the alcohol is ethanol.

5. The composition of claim 1 wherein the MPG is a poly-(1,3)-β-D-glucopyranosyl-(1,6)-β-D-glucopyranose.

6. A composition for treatment of a dermal injury including:
MPG in an amount of 0.05% to 0.15% (w/w) of the composition; and
ethanol in an amount of 5 to 15% (w/w) of the composition.

7. The composition of claim 6 wherein:
MPG is provided in an amount of 0.1% (w/w) of the composition; and
ethanol is provided in an amount of 10% (w/w) of the composition.

8. The composition of claim 6, further including one or more of a thickener, a pigment, and a surfactant.

9. The composition of claim 8 wherein the surfactant is triethanolamine.

10. The composition of claim 8 wherein the pigment is titanium dioxide.

11. The composition of claim 8 wherein the thickener is an acrylic acid polymer.

12. The composition of claim 11 wherein the polymer is Carbopol 980 NF.

13. The composition of claim 8 wherein:
titanium dioxide is provided as the pigment in an amount of 0.1% (w/w) of the composition;
carbopol 980 NF is provided as the thickener in an amount of 6% (w/w) of the composition;
triethanolamine is provided as the surfactant in an amount of 9% (w/w) of the composition.

14. The composition of claim 1 wherein the composition takes the form of a gel.

15. The composition of claim 14 wherein the gel has a water content of 80 to 90% (w/w) of the composition.

16. The composition of claim 15 wherein the gel has a water content of 85% (w/w) of the composition.

17. A method for treating a dermal injury including administering a composition including:
a micro-particulate glucan (MPG); and
a preservative in the form of an alcohol;
to a person in need thereof, wherein the MPG is provided in an amount effective for enabling the composition to induce wound healing or wound repair.

18. (canceled)

19. The composition of claim 6 wherein the composition takes the form of a gel.

20. The composition of claim 19 wherein the gel has a water content of 80 to 90% (w/w) of the composition.

21. The composition of claim 20 wherein the gel has a water content of 85% (w/w) of the composition.
